(19) United States
(12) Patent Application Publication
(10) Pub. No.: US 2024/0335312 A1
(43) Pub. Date: Oct. 10, 2024

(54) Knee support brace and method of wearing the same

(71) Applicant: Scott Geist, Hilton Head Island, SC (US)

(72) Inventor: Scott Geist, Hilton Head Island, SC (US)

(21) Appl. No.: 18/445,531

(22) Filed: Sep. 27, 2023

Related U.S. Application Data
(60) Provisional application No. 63/577,167, filed on Apr. 4, 2023.

(51) Int. Cl. A61F 5/01

(52) U.S. Cl. CPC A61F 5/0109

(57) ABSTRACT

The invention relates to an improved knee support brace and a method of wearing the same. In a preferred embodiment, the knee support brace comprises a central brace portion with a central aperture adapted to be placed over a knee of a person; a pair of encircling straps separately above and below on the central brace portion with an arcuate opening therebetween and made to extend in one direction from the central brace portion, each encircling strap having at least one fastening means affixed at their outer ends and at least one hook and loop fastener on an interior surface of the straps positioned to secure the central brace portion upon the knee of the person; and a tightening strap extending laterally between the pair of encircling straps on the central brace portion in a direction opposite to the encircling straps so that the tightening strap may wrap around the back of the knee and through the arcuate opening on the opposite side of the central brace portion and provide cross-fitting support to the knee of the person and prevent migration of the brace thereon. The central brace portion further includes pocket sleeves that accommodate a pair of removable hinges configured to be placed inside the knee support brace for providing lateral support to the knee.

### CROSS-REFERENCE TO RELATED APPLICATION

This application claims the benefit of U.S. Provisional Application Ser. No. 63/577,167 filed Apr. 4, 2023 for a Knee Support Brace and Method of Wearing Same.

### FIELD OF THE INVENTION

The present disclosure relates to orthotic devices of the type characterized as knee support braces and the construction and method of wearing same. More specifically, the present knee support brace incorporates a pair of adjustable hinges removably set on either side of the knee and a series of encircling straps that combine to prevent migration of the brace once set in place to provide maintain proper support during movement of the knee.

### BACKGROUND OF THE INVENTION

Knee pain is a very common problem faced by individuals during the course of their lives. Many knee issues are treated with medicines or physiotherapy. In addition, knee support braces m are often worn by individuals to assist in healing of knee injuries, serving to support and stabilize the knee cap, joints and muscles when they are weak, alleviating pain and enabling a patient to move around during recovery from any injury.

There are multiple patents on knee support brace devices that provide rigid or flexible panels along the brace with means for adjusting the brace to try and achieve an appropriate fit for the person wearing the same. However, it is extremely difficult to configure a brace with an ideal fit without causing discomfort to the person wearing it. Moreover, most of the braces provide adjustment means that wear out very quickly or provide a very clumsy fit around the knee thereby causing discomfort. One such recently granted patent, U.S. Pat. No. 10,709,593 issued Jul. 14, 2020, discloses a knee pain treatment apparatus that includes an upper member constructed to encircle a leg above a knee. The upper member includes an outer surface, an inner surface, an opening for exposing the front of the knee and a tensioning member integral with the upper member and depending downwardly therefrom. A patella biasing device is secured on the inner surface of the upper member and is arranged and constructed to border the opening. The apparatus further includes a flexible lower member arranged and constructed to encircle the leg below the knee and at least one tensioning member fastening system. The tensioning member fastening system is arranged and constructed for connecting the upper member and the lower member. When the upper member and lower member are mounted on the leg and connected, the tensioning fastening member may be stretched creating a downward biasing of the patella. While this and other prior art knee brace supports have served satisfactorily to support and stabilize the knee when fastened and fitted in place upon the wearer, those found in the prior art have not been found to adequately maintain a secure fit in relation to the knee size of a person and have evidenced migration of the knee brace from the fastened and fitted position reducing the support and stabilization intended.

In view of the above, there is a need for an improved knee support brace which can be worn for a long duration of time without discomfort and without migration from its fastened and fitted position upon the knee, which is easy to operate and fits appropriately to any individual regardless of the leg size.

### SUMMARY OF THE INVENTION

The inventive concepts presented herein are illustrated in a number of different embodiments, each showing one or more concepts, though it should be understood that, in general, the concepts are not mutually exclusive and may be used in combination even when not so illustrated.

Accordingly, in one aspect of the present invention provides a knee support brace. The knee support brace includes a central brace portion adapted to be placed over a knee of a person with a pair of encircling straps spaced apart above and below on the central brace portion, each encircling strap having at least one fastening means affixed at their outer ends wherein each of the pair of encircling straps include at least one hook and loop fastener on an interior surface of the straps and configured to secure the knee support brace to the knee of the person. The knee brace also includes a tightening strap positioned intermediately between the pair of encircling straps on the central brace portion, extending from an edge of a lateral surface of the central brace portion and opposite to an arcuate portion for providing support to the back portion of the persons/wearers knee, and a pair of removable hinges configured to be placed inside the knee support brace for providing support to the knee.

In an embodiment, the central brace portion of the knee support brace includes a first lateral pocket and a second lateral pocket disposed on each side of the central brace portion running longitudinally in a vertical direction thereupon for accommodating the pair of removable hinges, one on either side of the central brace portion. With the first and second lateral pockets thus vertically disposed, the pockets are intended to lie along and on each side of the approximate vertical center line of the wearer's knee when the knee support brace is in place with the hinges set in proper positions on either side of the knee.

In another embodiment, the present invention provides a method of wearing a knee support brace. The method includes the steps of adjusting a pair of members of a pair of removable hinges and inserting the hinges inside a pair of lateral reinforcing pockets on a central brace portion of the knee support brace wherein each of the pair of lateral reinforcing pockets are vertically disposed and are intended to lie along and on each side of the approximate vertical center line of the wearer's leg, then placing the central brace portion of the knee support brace on a patella of the knee of the person, before pulling a top encircling strap and a bottom encircling strap of a pair of encircling straps above and below the central brace portion in the same direction to adjust to the size of lower thigh and upper calf of the person. The method also includes the step of fastening the top and bottom encircling strap at an interior surface of the straps by a hook and loop fastener and at the outer ends of the top and bottom encircling strap through a fastening means affixed at the outer ends of the top and bottom encircling strap; and pulling and fastening a tightening strap intermediate of the top encircling and bottom encircling straps and in opposite direction thereto around the knee support brace for providing support to the back portion of the person/wearers knee.

In yet another embodiment, the present invention provides a knee support brace having a central brace portion adapted to be placed over a knee of a person, a pair of flexible encircling straps disposed apart above and below on the central brace portion, each having at least one fastening means affixed at their outer ends wherein each of the pair of encircling straps include at least one hook and loop fastener on an interior surface of the straps and configured to secure the knee support brace to the knee of the person; and a flexible tightening strap positioned between the pair of flexible encircling straps on the central brace portion extending from the edge of a lateral surface of the central brace portion and opposite to an arcuate portion for providing support to the back portion of the wearer's knee wherein inner edges of the encircling straps merge into an end edge having the arcuate portion or recess.

In an advantageous aspect, the pair of encircling straps and the tightening strap are formed of flexible and elasticized material enabling a secure fit of the knee brace on the wearer's leg. Further, at least one hook and loop fastener is positioned on the inside surface of the encircling strap for attaching to the central portion to securely conform to the knee, lower thigh and upper calf of the person/wearer.

To further clarify the advantages and features of the present invention, a more particular description of the invention will be rendered by reference to specific embodiments thereof, which are illustrated in the appended figures. It is appreciated that these figures depict only typical embodiments of the invention and are therefore not to be considered limiting of its scope.

### BRIEF DESCRIPTION OF THE DRAWINGS

The above contents of the present invention will become more readily apparent to those ordinarily skilled in the art after reviewing the following detailed description and accompanying drawings, in which:

FIG. 1 presents a layout view of the present knee support brace partially exploded to show a pair of hinges normally inserted in accordance with an embodiment of the invention;

FIG. 2 shows a perspective view of a hinge being removed from a sleeve of the knee support brace in accordance with an embodiment of the invention;

FIG. 2A shows a perspective front view of the hinge after removal from the sleeve of the knee support brace in accordance with an embodiment of the invention;

FIG. 2B shows a perspective view of a central hinge portion in an open position in accordance with an embodiment of the invention;

FIG. 2C shows a perspective view of the central hinge portion in open position with adjustment of the members in accordance with an embodiment of the invention;

FIG. 2D shows a perspective view of the hinge being reinserted after adjusting the members of the hinge to provide support on the lateral surface of the knee brace in accordance with an embodiment of the invention;

FIG. 3 shows a perspective view of the knee brace with hook and loop fastener on the inside surface of a top encircling strap of the knee brace in accordance with an embodiment of the invention;

FIG. 3A shows the knee support brace partially wrapped about the knee with an intermediate tightening strap extended before encircling the back of the knee to secure the knee brace upon the wearer in accordance with an embodiment of the invention; and

FIG. 3B shows a front view of the knee support brace wrapped in a closed assembly and worn about the knee in accordance with an embodiment of the invention.

Further, skilled artisans will appreciate those elements in the figures are illustrated for simplicity and may not have been necessarily drawn to scale. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having the benefit of the description herein.

### DETAILED DESCRIPTION OF THE INVENTION

Described herein are nonlimiting example embodiments of the present invention, which includes a back support brace and a method of wearing the same.

The various embodiments including the example embodiments will now be described more fully with reference to the accompanying drawings, in which the various embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, the sizes of components may be exaggerated for clarity.

It will be understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

Spatially relative terms, such as “central brace portion,” “encircling strap,” or “tightening strap,” and the like, may be used herein for ease of description to describe one element or feature's relationship to another element(s) or feature(s) as illustrated in the figures. It will be understood that the spatially relative terms are intended to encompass different orientations of the structure in use or operation in addition to the orientation depicted in the figures.

The subject matter of various embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Generally, the various embodiments including the example embodiments relate to a knee support brace and a method of wearing the same.

Referring to the drawings, the following is a list of numerical references to elemental parts and components of the present back support brace and those associated structural elements shown throughout the drawings and employed in connection with the present invention:
100 knee support brace;
101 central brace portion;
102 top encircling strap;
102a inner surface of the top encircling strap;
102b inner edge of the top encircling strap;
103 bottom encircling strap;
103a inner surface of the bottom encircling strap;
103b inner edge of the bottom encircling strap;
104 fastener pads affixed at outer ends of the encircling straps;
105 fastener patches on inside surface of the encircling straps;
106 intermediate tightening strap;
106a fastener pad;
107 side edge of central brace portion;
108 arcuate opening;
109, 110 first and second removable hinges;
109a, 109b movable arms of first removable hinge;
110a, 110b movable arms of second removable hinge;
109c,110c central hinge members;
111, 112 pockets for accommodating the removable hinges;
113 vertical center line of wearers leg;
114 C-shaped edge portion; and
115 flattened cutout.

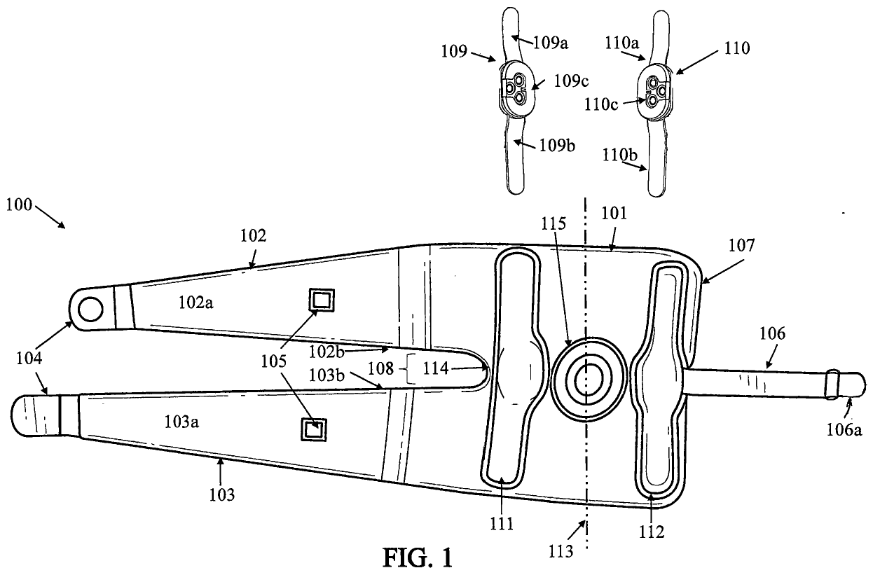

Referring initially to FIG. 1, the present knee support brace 100 is shown in a layout configuration viewed from the front or outer surface thereof and constructed in accordance with a preferred embodiment of the invention. The knee support brace 100 includes a central brace portion 101 having a pair of encircling straps 102, 103 extending from one side of the central brace portion, with the encircling straps being spaced apart in their respective extensions, the top strap 102 being above and the bottom strap 103 below on the central brace portion 101. Each of the pair of encircling straps 102, 103 has at least one fastening means 104, typically a hook and loop fastener pad, affixed at their respective outer ends on the interior surface of the straps with an additional hook and loop fastener patch 105 positioned along the interior surface of each encircling straps to provide secure engagement of the straps around and onto the exterior surface of the central brace portion 101 when the knee support brace 100 is being placed upon the knee of the wearer.

The knee support brace 100 further includes a tightening strap 106 extending from a side edge 107 of the central brace portion 101 oppositely and away from the encircling straps 102, 103 on the other side of the central brace portion with the tightening strap extending substantially from the middle of the edge of central brace portion in an intermediate position between those of the separate encircling straps extending from the opposite side of the central brace portion. Like those of the encircling straps 102, 103, the tightening strap 106 extending away from the side edge 107 of the central brace portion 101 is provided at its end with a fastener pad 106a, typically made of a hook and loop fastener material, intended to engage and attach the tightening strap to the opposite side of the central brace portion 101 around the rearward side thereof and then forward through an arcuate opening 108 between the encircling straps. This path of engagement of the tightening strap 106 to the central brace portion 101 through the arcuate opening 108 on the opposite side provides firm support to the back portion of the wearer's knee and prevents migrating movement of the present knee support brace 100 when secured in place.

The present knee support brace 100 further includes a pair of removable hinges 109, 110 configured to be placed inside sleeve-like pockets 111, 112 constructed and formed along either side of the central brace portion 101. The first sleeve-like pocket 111 and a second sleeve-like pocket 112 are similarly constructed having an opening through which a respective one of the hinges 109, 110 may be inserted and a chamber formed therein that is sized in length and width to accommodate and hold the respective hinges in place. In their position on either side of the central brace portion 101, the sleeve-like pockets 111, 112 are vertically disposed and intended to lie along and on each side of the approximate vertical center line 113 of the braced leg and knee of the wearer. A patella opening formed centrally within a flattened cutout 115 in the central brace portion 101 substantially aligns with the vertical center line 113 and is located approximately midway between the first and second lateral sleeve-like pockets 111, 112. The flattened cutout 115 provided in the central brace portion may be of different shapes including circular, oval, rectangle, or rhombus.

The encircling straps 102, 103 are each preferably of sufficient length to encircle the entire thigh or calf, as the case may be, at least once. In the space separating the encircling straps 102, 103, respective inner edges 102b and 103b of the encircling straps merge into a C-shaped edge 114 that borders arcuate opening 108. The arcuate C-shaped edge portion 114 and the arcuate opening 108 formed thereby between the encircling straps 102, 103 and on the side of the central brace portion 101 provides the engagement access for the tightening strap 106 and is shaped to provide the tightening strap with substantially smooth close fit around the knee when fastened. In a related aspect of the present embodiment of the invention, the various edges of the encircling straps 102. 103 and tightening strap are stitched or glued to form a seam around the edge for enabling proper fit of the knee brace around the knee of the wearer. In a further embodiment, the pair of encircling straps 102, 103 and the tightening strap 106 are made of elastic material permitting reasonable length adjustments of the straps depending on the size of the wearer's leg and knee being braced.

Referring further to FIG. 1, the present knee support brace 100 provides the central brace portion 101 that is formed to rest in support of a knee cap of a person. The central brace portion 101 and its sleeve-like pockets 111, 112 accommodate the removable hinges 109, 110 to provide flexible side support to the knees when braced. Each of the pair of removable hinges 109, 110 includes a pair of moveable members 109a, 110a connected to a central hinge portion 109b, 110b having means to adjust and set the proper angle of the moveable members 109a, 110a in relation to the knee to ensure an optimal fit for the person.

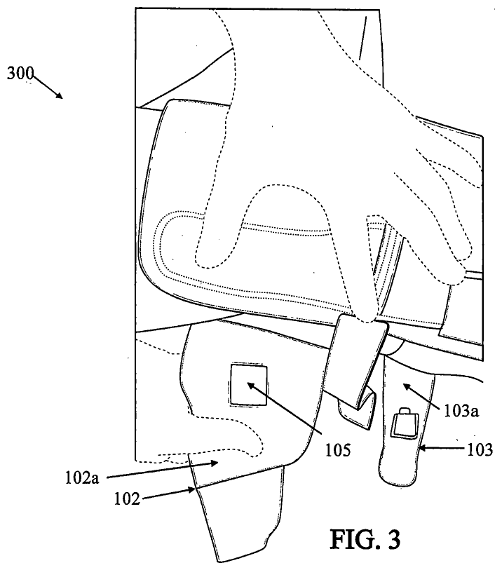

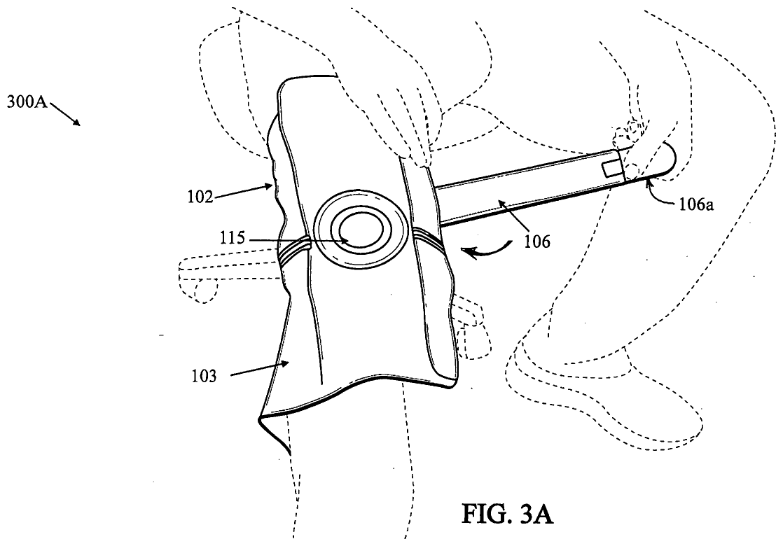

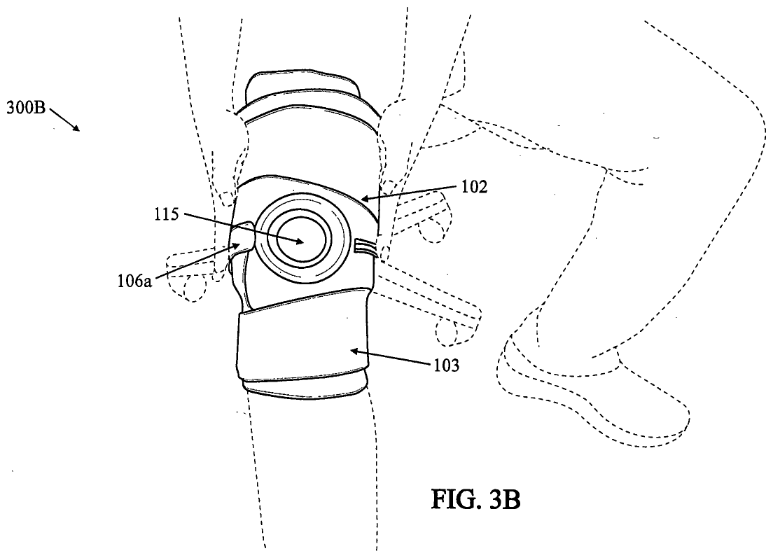

Referring to FIGS. 3A and 3B in conjunction with FIG. 1, the encircling straps 102, 103 include an upper strap 102 and a bottom strap 103 that fasten above and below on the central brace portion 101 in one direction with the fastener pads 104 at the end of each circling strap and the fastener patches 105 along the inside surfaces thereof engaging the outer surfaces of the central brace portion, thus holding the central brace with some degree of compression upon the knee, as best seen in FIG. 3A. With the central brace portion 101 thus held upon the knee, the tightening strap 106, seen held in its intermediate position on the central brace portion 101 in FIG. 3A, is swung around the back of the knee and to the rear of the central brace portion 101 in the opposite direction to those of the encircling straps 102, 103 to engage and fasten to the outer surface of the central brace portion on the opposite side using fastener pad 106a accessed through the arcuate opening 108. The combination of these cross fastening straps 102, 103 and 106 about and upon the central brace portion 101 provides firm and secure support for the knee in the present support brace 100 and prevents migration of the brace during usage.

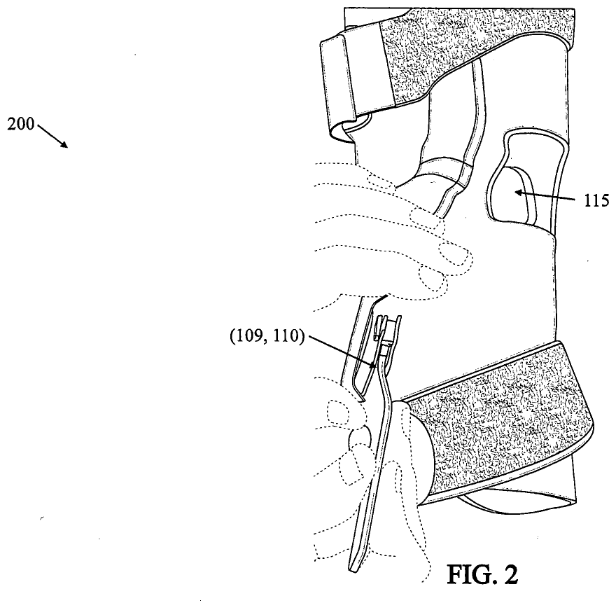

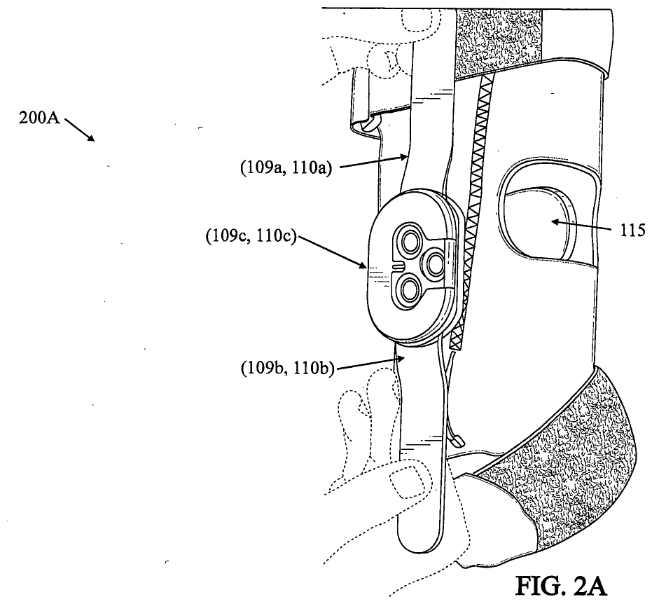

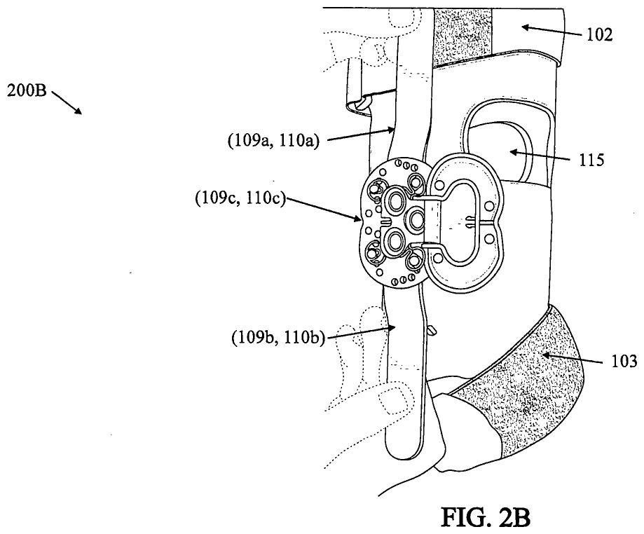

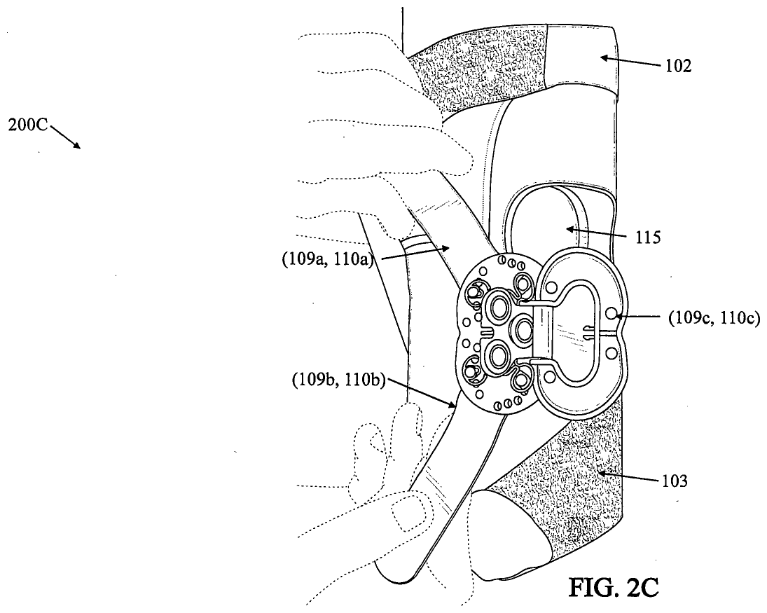

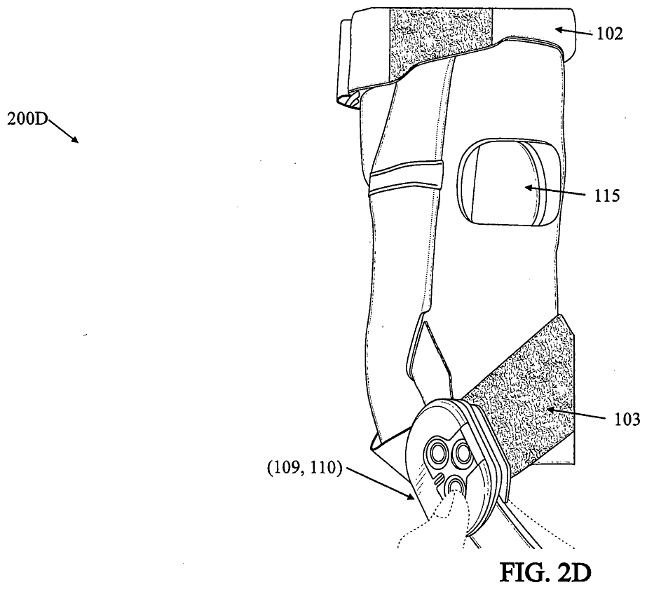

Referring now to FIGS. 2 and 2A in conjunction with FIG. 1, perspective views 200, 200A of an associated portion of the central brace portion 101 are shown with the hinges 109, 110 being removed are shown in accordance with an aspect of the present knee support brace 100. In this aspect of the present embodiment, the invention provides a improved method of wearing the knee support brace with adjustable lateral support provided by adjustment of the hinges. Each of the hinges 109, 110 include movable arm members 109a, 109b and 110a, 110b made to extend in opposite directions from a central hinge member 109c, 110c used to control the positional angles of the arm members on each hinge. Using the controls of the central hinge members 109c, 110c to adjust the support angles of the movable arm members 109a, 109b and 110a, 110b on the respective hinges 109, 110 in a manner most suitable for the individual wearer of the knee support brace will benefit the wearer when the hinges are positioned in place within the pockets 111, 112 on either side of the central brace portion 101. As seen in the respective views thereof 200B, 200C, 200D shown in FIGS. 2B, 2C and 2D, each hinge 109, 110 may be removed from the associated pockets 111, 112 to adjust and set the angular position most suitable for the wearer of the present knee brace support 100 before reinserting the hinges back into the pockets of the central brace portion 101 of the knee support brace.

The knee support brace 100 may include a lateral reinforcing means in support of the pockets 111, 112 holding the respective hinges 109, 110, with the lateral reinforcing means being vertically disposed along the central brace portion 101 and intended to lie along and on each side of the approximate vertical center line 113 of the wearer's leg. The method of wearing the knee brace includes placing the central brace portion 101 of the knee support brace 100 on the patella of the knee of the person, pulling the top encircling strap 102 and the bottom encircling strap 103 of a pair of encircling straps above and below the central brace portion 101 in the same direction to adjust to the size of lower thigh and upper calf of the person. The method further includes fastening the top 102 and bottom 103 encircling straps at interior surface 102a of the straps by a hook and loop fastener 105 and at outer ends of the top and bottom encircling strap through a fastening means 104 affixed at outer ends of the top and bottom encircling strap, and then pulling and fastening the tightening strap 106 intermediate of the top encircling strap 102 and bottom encircling strap 103 in opposite direction thereto around the knee support brace for providing support to back portion of the person/wearers knee.

FIG. 3 shows a perspective view 300 of the knee brace support with hook and loop fastener patch 105 on the inside surface of the top encircling strap 102 of the knee brace. A similar patch 105 may be positioned and affixed on the inside surface of the lower encircling strap 103. As referred to above, FIG. 3A shows view 300A of a tightening strap 106 of the knee brace for securing the knee brace to the knee of the wearer. Further, FIG. 3B shows a front view 300B of the knee support brace worn by a person in accordance with an embodiment of the invention.

While various elements of the present knee support brace 100 including the encircling straps 102, 103 and tightening strap 106, are shown to be attached or connected through a hook and loop fastener material, such as VELCRO, at the outer ends, it shall be apparent to a person skilled in the art that other connections may be used for securing these elements to the surface of the knee brace within the scope of this invention.

Variations of the type, form, size, and material of any of the central brace portion, top encircling strap, bottom encircling strap, and hinges may be of any of those described previously and used in any of the methods described.

Obviously, other embodiments and modifications of the present invention will readily come to those of ordinary skill in the art having the benefit of the teachings presented in the foregoing description and drawings. Alternate embodiments of different shapes and sizes, as well as substitution of known materials or those materials which may be developed at a future time to perform the same function as the present described embodiment are therefore considered to be part of the present invention. Furthermore, certain modifications to the described embodiment that serve to benefit its usage are within the scope of the present invention. Accordingly, it is understood that this invention is not limited to the particular embodiment described, but rather is intended to cover modifications within the spirit and scope of the present invention including the appended claims.

What is claimed is:

1. A knee support brace, comprising:
a central brace portion adapted to be placed over a knee of a person;
a pair of encircling straps spaced apart and made to extend from one side of the central brace portion with an arcuate opening therebetween, the encircling straps extending laterally in a direction above and below the position of the knee, each encircling strap having at least one fastening means affixed at the outer ends thereof and at least one hook and loop fastener on an interior surface thereof for wrapping the central brace portion upon the knee of the person; and
a tightening strap extending laterally from one side of the central brace portion between the pair of encircling straps and in an opposite direction thereto, the tightening strap adapted to wrap around the back of the knee and through the arcuate opening between the encircling straps and fasten onto the opposite side of the central brace portion thereby providing cross-fitting support to the knee and preventing migration of the brace thereon.

2. The knee support brace of claim 1, further comprising;
a pair of removable hinges configured to be placed inside the central brace portion for providing lateral support to the knee.

3. The knee support brace of claim 2, wherein the central brace portion includes a first pocket sleeve and a second pocket sleeve vertically disposed on the central brace portion for accommodating the pair of removable hinges in positions along and on each side of the approximate vertical center line of the leg of the person.

4. The knee support brace of claim 3, wherein the central brace portion further comprises a patella opening located approximately midway between the first and second pocket sleeves.

5. The knee support brace of claim 3, wherein the hinges includes a central hinge portion having means to position a pair of movable members at the appropriate angle in relation to the knee of the person.

6. The knee support brace of claim 5, wherein the pair of encircling straps and the intermediate tightening strap are disposed and arranged in a cross-fitted connection to the central brace portion to enable a secure and stable fit of the knee brace on the leg without migration therefrom.

7. The knee support brace of claim 6, wherein at least one hook and loop fastener is positioned on the inside surface of the encircling strap for attaching to the central brace portion to securely conform to the knee, lower thigh and upper calf of the person.

8. A method of wearing a knee support brace upon a person, the method comprising:
adjusting a pair of removable hinges and inserting the hinges inside a pair of lateral sleeve pockets on a central brace portion of the knee support brace along and on each side of the approximate vertical center line of the leg of the person;
placing the central brace portion of the knee support brace on a patella of the knee of the person;
routing a pair of encircling straps spaced apart and extending from the central brace portion in the same direction to enwrap the lower thigh and upper calf of the person;
fastening the encircling straps at an interior surface of the straps by a hook and loop fastener and at outer ends of the top and bottom encircling strap through a fastening means affixed at the outer ends of the encircling strap;
wrapping a tightening strap between the encircling straps from the opposite side of the central brace portion in opposite direction thereto; and
fastening the tightening strap between the encircling straps and onto the opposite side of the central brace portion to provide cross-fitting support to the knee of the person and prevent migration of the brace thereon.

9. The method of claim 8, wherein the central brace portion provides a patella opening located approximately midway between the pair of lateral sleeve pockets.

10. The method of claim 8, wherein the pair of encircling straps and the intermediate tightening strap are disposed and arranged in a cross-fitted connection to the central brace portion to enable a secure and stable fit of the knee brace on the leg without migration therefrom.

11. The method of claim 8, wherein the hook and loop fasteners are positioned on the inside surface of the encircling straps for attaching to the central portion to securely conform to the knee, lower thigh and upper calf of the person.

12. A knee support brace, comprising:
a central brace portion formed with a central opening adapted to be placed over a knee of a person;
a pair of encircling straps spaced apart above and below on the central brace portion and extending from a side edge thereof in a first direction, each encircling straps having at least one fastening means affixed at the outer ends thereof and at least one hook and loop fastener on an interior surface of the straps positioned to secure the central brace portion to the knee of the person; and
an intermediate tightening strap laterally disposed between the pair of encircling straps and extending from an opposite side edge of the central brace portion in an opposite direction to the encircling straps, so that the tightening strap may be wrapped around the back of the knee and through the space between the encircling straps and fasten onto the opposite side of the central brace portion thereby providing cross-fitting support to the knee and preventing migration of the brace thereon.

13. The knee brace of claim 12, further comprising:
a pair of removable hinges adjustable in configuration and placed inside the central brace portion on either side of the central opening for providing lateral support to the knee wherein angle of the hinge is adjustable before the hinge is inserted inside the knee support brace.
